(19) United States
(12) Patent Application Publication
(10) Pub. No.: US 2025/0144143 A1
(43) Pub. Date: May 8, 2025

(54) Virally Educated T cells

(71) Applicant: ImmunityBio, Inc., San Diego, CA (US)

(72) Inventor: Patrick Soon-Shiong, Culver City, CA (US)

(73) Assignee: ImmunityBio, Inc., San Diego, CA (US)

(21) Appl. No.: 18/938,864

(22) Filed: Nov. 6, 2024

Related U.S. Application Data
(60) Provisional application No. 63/596,583, filed on Nov. 6, 2023.

(51) Int. Cl. A61K 35/17; A61K 38/00; A61K 39/00; C07K 14/005; C07K 14/54; C12N 15/86

(52) U.S. Cl. CPC A61K 35/17; A61K 40/11; A61K 40/46; C07K 14/005; C07K 14/5443; C12N 15/86; A61K 38/00; C07K 2319/06; C12N 2710/10043; C12N 2770/20022

(57) ABSTRACT

Compositions and methods are described for a treatment of enduring viral infection in general, and long COVID in particular. The composition comprises T cells, antigen presenting cells (APC), and optionally NK cells derived from the whole blood of a patient. The APC are genetically engineered to comprise nucleic acids encoding antigenic peptide sequences for MHC-complexed cell surface expression, wherein further exposure to an IL-15 agonist and patient T cells yields activation and expansion of virally-educated T cells for re-administration to the patient, whereby cells harboring the virus are targeted for T-cell mediated cytotoxicity.

This application claims priority to our copending US Provisional application with the Ser. No. 63/596,583, which was filed Nov. 6, 2023, and which is incorporated by reference herein in its entirety.

### SEQUENCE LISTING

The content of the XML of the sequence listing named 102690.0067PCT.XML, which is 5,415 bytes in size was created on Nov. 5, 2024 and electronically submitted via EFS-Web along with the present application, and is incorporated by reference in its entirety.

### FIELD OF THE INVENTION

The field of the invention relates to compositions and method for treatment of viral disease, especially as it relates to treatment of a subject using ex vivo modified apheresis product.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Long Covid, also known as Post-Covid Conditions (PCC), refers to the wide range of symptoms and conditions that some people experience four or more weeks after an initial infection by SARS-COV-2, the virus that causes Covid-19. The symptoms and conditions, which may last for weeks, months, or years, can be persistent (meaning they developed during an acute Covid-19 illness and haven't gone away), recurrent (meaning they may go away after the initial illness then return), or new (meaning they were not present initially but developed later).

Symptoms of Long Covid can range in severity from mild to debilitating. Some people report that Long Covid symptoms prevent them from returning to work or school, and others have difficulties performing everyday chores or even walking short distances.

Long Covid most often occurs in people who had severe Covid-19 illness, but it is not restricted to those who were critically ill or hospitalized. People with mild disease and even those who did not develop symptoms can also be affected. In fact, most people with Long Covid had mild acute Covid-19. This is because many more people have mild Covid-19 than severe Covid-19, so even if a small percentage of them develop Long Covid, they nevertheless account for the majority of Long Covid cases. Adults and children can both be affected, though Long Covid appears to be more common in adults. Researchers do not yet know how common the condition is, but studies have estimated that it occurs in 5% to 30% of people with Covid-19.

In addition to PCC, Long Covid goes by many other names, including long-haul Covid, post-acute sequelae of Covid-19 (PASC), long-term effects of Covid-19, and chronic Covid, among others.

It is not yet understood if the symptoms of Long Covid result from the initial infection or are a manifestation of an enduring infection. Viral persistence is suggested by the fact that viral DNA and RNA have been found up to two years after acute infection in some patients. Also, SARS-COV-2 can reactivate other viruses such as endogenous retroviruses and EBV which are dormant. Currently there is no treatment for long Covid, and patients are usually treated for their symptoms only.

Thus, there remains a need in the art for new compositions and methods for treating Long Covid.

### SUMMARY OF THE INVENTION

The inventive subject matter is directed to various compositions and methods of viral vaccine compositions. Disclosed herein are pharmaceutical compositions for use in a patient having a viral infection, the composition comprising IL-15 or an agonist derivative thereof; and viral-educated T cells. The IL-15 agonist derivative may comprise IL-15: IL: 15RaSu and/or a stabilized derivative, such as an Fc domain conjugate, HCW-9218,0 or N-803. Also disclosed herein are methods of making and using such compositions.

The viral educated T-cells comprise T-cells that are isolated from whole blood of the patient and exposed ex vivo to dendritic cells, wherein the dendritic cells are 1) isolated from whole blood of the patient and 2) transfected with a nucleic acid vector encoding at least one antigenic viral peptide sequence. In some embodiments, the composition further comprises memory-like cytokine enhanced natural killer (m-ceNK) cells, wherein the m-ceNK cells are isolated from the whole blood of the patient. The autologous T cells and dendritic cells are preferably derived from an apheresis product of the patient. In some embodiments, the T cells are ex vivo exposed to dendritic cells in the presence of IL-15 or an agonist derivative thereof, or in the presence of mesenchymal stem cells (MSC).

In preferred embodiments, the nucleic acid vector comprises an adenoviral (Ad) vector encoding antigenic viral peptides such as SARS-COV-2 Spike(S) and nucleocapsid (N) proteins. In one embodiment, the Ad vector comprises a first nucleic acid portion and a second nucleic acid portion; wherein the first nucleic acid portion encodes a CoV2 spike(S) protein having the amino acid sequence of SEQ ID NO:3; and wherein the second nucleic acid portion encodes a chimeric protein comprising 1) a CoV2 nucleocapsid (N) protein having the amino acid sequence of SEQ ID NO: 1 and 2) an endosomal targeting sequence (ETSD) having the amino acid sequence of SEQ ID NO: 2. Alternatively, the nucleic acid vector comprises an RNA vector. In some cases, the viral peptide sequence is derived from SARS-COV-2, HPV, or HIV.

Further disclosed herein are use of the composition in the preparation of a medicament for the treatment of viral infection. In preferred embodiments, the viral infection comprises SARS-COV-2, HPV, or HIV. In still preferred embodiments, the viral infection comprises long Covid.

In another aspect, disclosed herein is a method of treatment of long Covid in a patient in need thereof, the method comprising: transfecting dendritic cells isolated from whole blood of the patient with a nucleic acid vector encoding at least one antigenic peptide derived from SARS-COV-2; exposing T cells, isolated from whole blood of the patient, to the dendritic cells, thereby expanding the T cells into viral educated T cells; and treating long Covid by administering IL-15 or an agonist derivative thereof and the viral educated T cells to the patient.

In some embodiments, the nucleic acid vector comprises an adenoviral (Ad) vector, and preferably an Ad vector comprising an E1 gene region deletion and an E2b gene region deletion. Preferably, the adenoviral (Ad) vector comprises a nucleic acid portion that encodes a SARS-CoV2 S protein, and a nucleic acid portion that encodes a chimeric protein comprising a SARS-CoV2 N protein and an endosomal targeting sequence.

The IL-15 agonist derivative may comprise IL-15:IL:15RaSu and/or a stabilized derivative, such as an Fc domain conjugate, HCW-9218, or N-803. Preferably, the T cells and dendritic cells are derived from an apheresis product of the patient. Upon transfection of the dendritic cells by the nucleic acid vector, the dendritic cells are exposed to the T cells in the presence of IL-15 or an agonist derivative thereof to thereby produce the viral educated T cells. The viral educated T cells may be administered to a long Covid patient intravenously for treatment of the disease. In some embodiments, IL-15 or agonist derivative thereof may also be administered, either simultaneously or separately. In that regard, it should be recognized that the virally educated T cells and the IL-15 or agonist derivative thereof may be formulated together or formulated separately. Optionally, Natural Killer (NK) cells are administered to the patient. The NK cells may comprise m-ceNK cells, NK-92 cells, aNK cells, haNK cells, and/or t-haNK cells.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an exemplary schematic flow chart of a viral disease treatment according to the inventive subject matter.

FIG. 2 illustrates that DC-driving cytokines lower overall background and favor specificity in the DCs.

FIG. 3 illustrates the role of GM-CSF and FLT3L superkines in generating DCs.

FIG. 4 illustrates the effects of cytokine cocktails on antigen specific T-cell expansion.

FIG. 5 illustrates the use of AdV to deliver antigen with poly-L-lysine.

### DETAILED DESCRIPTION

The inventors have disclosed herein pharmaceutical compositions for treating a viral disease comprising IL-15 or an agonist derivative thereof (such as N-803) and viral-educated T cells. In preferred embodiments, the viral disease is long Covid.

The inventors have found that N-803 is effective in treating viral diseases, and especially latent viral diseases. N-803 induces expression of latent HIV antigens in patients with chronic HIV infection. The inventors further saw a similar effect of N-803 with latent SARS-COV-2 infection in patients with latent covid. Expression of antigens on antigen presenting cells (APC) are then used to stimulate T cells which express T cell receptor (TCR) that are specific for APC MHC-bound viral antigens.

In one aspect of the present disclosure, T cells and antigen presenting dendritic cells are isolated from whole blood of a patient (apheresis). The dendritic cells are then transfected with a nucleic acid vector encoding at least one viral antigen. These transfected and modified dendritic cells are exposed to the T cells that were isolated from whole blood of the patient. The T cells expand in number upon exposure to the dendritic cell. This population of T cells that are exposed to one or more viral antigens are referred to as viral-educated T cells throughout the present disclosure. In one embodiment, the nucleic acid vector is an adenoviral vector, for example human adenovirus serotype 5 (hAd5). The hAd5 vector may be replication defective, for example by a deletion of the early 1 [E1] and early 2b [E2b] genes.

In preferred embodiments, the viral antigen comprises one or more SARS-COV-2 antigens, for example the SARS-COV-2 Spike(S) and nucleocapsid (N) proteins. The SARS-COV-2 antigens are further described in US Patent Appl. No.: 16/883,263, PCT Publication No.: WO/2021/183665, PCT Publication No.: PCT/IB2021/054887, PCT Publication No.: WO2023/102375, each of which is incorporated by reference in its entirety, including the figures and sequences.

The viral antigen is preferably encoded in a replication defective adenovirus (hAd5 -E1, -E2b). The viral antigen is selected from the group consisting of coronavirus 2 (CoV2) nucleocapsid protein, CoV2 spike protein, and a combination thereof. The CoV2 nucleocapsid protein is contemplated to have at least 85% identity, at least 90% identity, at least 95% identity, or 100% identity to SEQ ID NO:1. In some cases, the CoV2 nucleocapsid protein may conjugated with an endosomal targeting sequence (ETSD) having at least 85% identity, at least 90% identity, at least 95% identity, or 100% identity to the amino acid sequence of SEQ ID NO: 2. The CoV2 spike protein is contemplated to have at least 85% identity, at least 90% identity, at least 95% identity, or 100% identity to SEQ ID NO:3.

Optionally the expansion step is performed in the presence of an IL-15 agonist such as N-803, wherein the MHC-complexed expression of latent viral peptides is stimulated. The virally educated and expanded T cells are then formulated for re-administration to the patient, along with an IL-15 agonist such as N-803.

Thus, in one embodiment, the inventors disclose pharmaceutical compositions comprising virally educated T cells and N-803. The inventors envision that the co-administration of an IL-15 agonist (such as N-803) with viral educated T cells to long-covid patients results in reversal of latency allowing the body's immune system to attack the virus. Thus, in other words, co-administration of virally educated T cells and N-803 resulted in a synergistic effect that was not seen with either one of the individual components when administered alone, and thus is more than just an additive effect.

With regards to the cytokine IL-15, all forms of IL-15 including IL-15 superagonist, IL-15 mutant, IL-15 receptor alpha (IL-15Rα) complex (IL-15:IL-15Rα), fusion proteins comprising IL-15, or combinations thereof may be used in the composition disclosed herein. Stabilized IL-15 includes IL-15 superagonist such as nogapendenkin alpha imbakicept (Alt-803, N-803, Vesanktiva), as well as stabilized IL-15:IL-15Rα fusion protein. The IL-15:IL-15Rα fusion protein is an IL-15^{N72D}:IL-15RαSu/Fc complex (ALT-803, also known as nogapendekin alfa-inbakicept) comprising a dimeric IL-15RαSu/Fc and two IL-15^{N72D} molecules. U.S. patent application Ser. No. 16/985,728, U.S. patent application Ser. No. 16/505,528, U.S. Pat. No. 11,351,196, U.S. Patent Application Ser. No. 63/156,269 U.S. Pat. No. 8,163,879, U.S. Pat. No. 8,507,222, and U.S. Pat. 10,537,615, discuss IL-15 and its derivatives more broadly and are herein incorporated by reference in its entirety.

In some embodiments, the composition comprising N-803 and viral-educated T cells further comprises cytokine-enhanced NK cells (ceNK cells). The ceNK cells disclosed herein refers to NK cells in which the cytotoxic activity is enhanced by cytokine stimulation. ceNK cells are prepared by inducing NK cells with a corticosteroid and optionally a cytokine composition comprising IL-15, IL-15:IL-15Rα, or agonist derivatives thereof, such as N-803. The cytokine composition may comprise a fusion protein, wherein the fusion protein comprises IL-15 or an agonist derivative thereof. Fusion proteins comprising IL-15, wherein the fusion protein has increased stability over IL-15 are preferred. While not limiting the inventive subject matter, it is generally preferred that the corticosteroid is hydroxycortisone and the optional cytokine is N-803.

The Memory-like Cytokine-Enhanced NK Cells (m-ceNK) disclosed herein comprises enriched and expanded NK cells obtained from peripheral blood of donors using the apheresis technique to generate NK cells with a memory-like phenotype. The m-ceNK cells exhibit both high cytotoxicity and increased interferon-gamma production. These m-ceNK cells can be generated from an individual donor for autologous cell therapy or may be generated as an allogeneic product from cord blood. In addition to having enhanced efficacy, m-ceNK cells can be infused easily in an outpatient setting.

As a non-limiting example, the m-ceNK cells may be generated by a step of obtaining a plurality of mononuclear cells and contacting the plurality of mononuclear cells with a corticosteroid and optionally a cytokine. In another step, the plurality of mononuclear cells are incubated in the presence of the corticosteroid and the optional cytokine to enrich the mononuclear cells in NK cells, and the enriched NK cells are then induced with a cytokine composition comprising IL-15, IL-12, and IL-18, or agonist derivatives thereof. The composition may comprise one or more fusion proteins, wherein the fusion proteins comprise at least one of the IL-15, IL-12, and IL-18 cytokines, or agonist derivatives thereof. The cytokine composition may comprise a T×M fusion protein to generate the m-ceNK cells, wherein the T×M fusion protein comprises a protein portion having IL-12 activity, a protein portion having IL-15 activity, and a protein portion having IL-18 activity.

Further description of making the m-ceNK cells and its advantageous properties is described in PCT/US2022/018290, which is incorporated by reference in its entirety. U.S. patent application Ser. No. 17/375,985 and U.S. Pat. No. 11,453,862 provide additional alternative methods of inducing enrichment and expansion of NK cells. Each of the above references are incorporated by reference in its entirety.

In some embodiments, prior to the transfection of dendritic cells with viral antigenic peptides, the viral antigenic peptides are selected and the molecular nature of the viral infection is confirmed by PCR, for example a hand-held nudge device. Confirmation of the virus informs the peptide selection, which can be done through various strategies known in the art. Peptides are selected for various properties including size and MHC binding (depending on the HLA type of the patient). Nucleic acids encoding the antigenic peptides are added to the dendritic cells by various means known in the art. Adenoviral vectors may be used. RNA, optionally mRNA, can be complexed with a lipid nanosphere or a mannan nanogel.

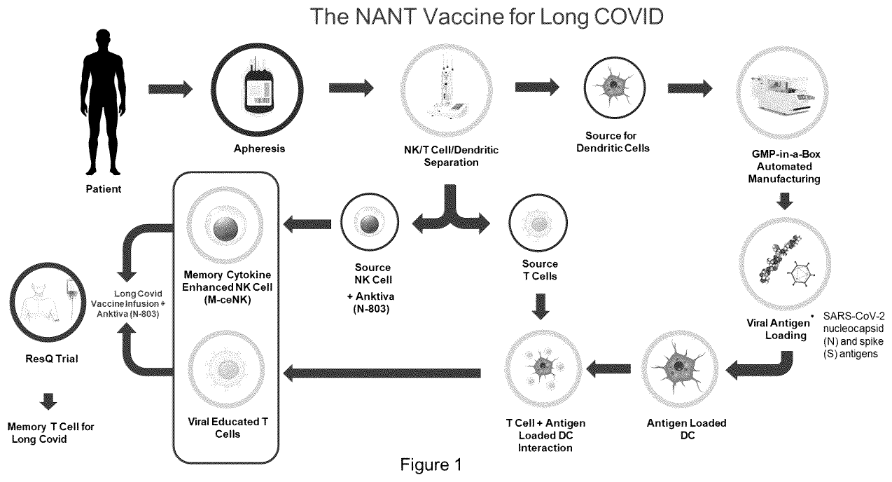

A preferred embodiment of the disclosure is a method of treatment for long Covid, wherein latent low level viral expression may cause the varying symptoms associated with long Covid. IL-15 is used in two aspects of the method. First it is used in the preparation of the virally educated T cell, as T cells are exposed to dendritic cells under IL-15 stimulation which has been shown to stimulate T cell expansion as well as antigen presenting cell cell-surface expression of MHC-presented antigenic peptides. Second, the IL-15 is administered along with the educated T cells to stimulate said T cells but also to stimulate expression of latent COVID antigens. This is known as the “kick” phase of a “kick and kill” strategy of provoking expression of latent virus, thus enabling targeting by cytotoxic lymphocytes. The method is illustrated in FIG. 1.

While long Covid is an exemplary use of this disclosure, the compositions and methods disclosed herein may be used for any other viral disease, such as HIV, influenza, HPV, EBV, etc

Additional therapeutic agents that are contemplated to be included in the compositions and methods disclosed herein include m-CENK, NK-92 cells (hank, t-haNK), antiviral drugs, bNAbs, and HDAC inhibitors.

m-ceNK comprise memory-like cytokine-enhanced natural killer cells (PCT/2021/006876). They can be derived from the patient whole blood by methods known in the art, and autologously administered. m-ceNK cells are characterized by enhanced cytotoxicity against tumor cells or infected cells. In an embodiment, the m-CENK can be expanded under a composition comprising at least one of a corticosteroid such as dexamethasone, IL-15, IL-18, IL-12, an anti-CD16 Ab, and optionally an anti-CD3 Ab.

In especially preferred aspects, apheresis can be used to prepare various cell fractions disclosed herein for treatment of the same patient in a concurrent or serial manner.

In one typical example, as schematically illustrated in FIG. 1, a subject diagnosed with a viral disease (for example, long Covid) is optionally treated with N-803 to stimulate T-cell and NK-cell production prior to apheresis during which PBMCs are collected. Most typically, the PBMCs will include a variety of immune cells such as CD4⁺ T cells, CD8⁺ T cells, B cells, macrophages, dendritic cells, Kupffer cells, etc. The NK cells, T cells, and dendritic cells may be separated from each other. Alternatively, the complete PBMC preparation may be transfected with an hAd5 encoding one of more viral antigens, wherein the DC specifically take up the Ad5 vectors. The dendritic cells and/or macrophages are used for infection with a viral antigen leading to antigen presentation of the recombinant antigens on antigen presenting cells such as macrophages and dendritic cells. In preferred embodiments, viral antigens are encoded by a recombinant adenovirus, such as hAd5. The virally loaded dendritic cells that present the recombinant neoantigens on the MHC class I and/or class II complexes are then contacted with T cells (including unlicensed T cells, immature and mature unprimed T cells, CD4⁺ T cells, and CD8⁺ T cells), which in turn triggers development (activation and expansion) of antigen specific cytotoxic and helper T cells. Of course, it should be recognized that the infection of the antigen presenting cells with the viral antigen may be performed in the presence of immune stimulating cytokines (IL-2, IL-15, etc.), co-stimulatory molecules, and/or checkpoint inhibitors.

In especially contemplated compositions and methods, it is generally preferred that the T cells are exposed to the antigen loaded dendritic cells for a time sufficient to induce measurable T cell expansion, and particularly clonal expansion of antigen-responsive T cells. As will be recognized, such expansion may be stimulated by addition of IL15, IL15:IL15Rα or an IL15Rβγ agonist derivative thereof, IL15:IL15RαIgG4Fc, HCW-9218, or N-803. HCW-9218 is further described in U.S. Pat. No. 11,518,792, which is incorporated by reference herein in its entirety.

In further preferred aspects, the dendritic cells are exposed to the viral antigen for a time sufficient for the dendritic cells to take up substantially all of the viral antigen. Viewed from a different perspective, upon ex vivo infection, it is typically preferred that the viral titer in the antigen presenting cells fraction of the PBMC is low to undetectable, meaning that substantially all the virus has been taken up by the antigen presenting cells (APC). Such time can be empirically ascertained by quantification of viral infectivity in the cell composition (e.g., using hexon assay where the virus is an adenovirus). Once T cell activation and/or expansion is observed and viral titer in the supernatant is low to undetectable, the so prepared cell suspension can then be used for infusion. Thus, by using contemplated methods and compositions, it should be appreciated that the infection of antigen presenting cells with the therapeutic viral antigen is optimized while minimizing or entirely avoiding potential adverse effects due to off-target virus binding. The inventors also contemplate that the time of ex-vivo exposure be determined by MHC presentation of Ad-encoded peptides, wherein intravenous (IV) administration of Ad-treated apheresis product results in subsequent in vivo stimulation of antigen-responsive CD4⁺ and CD8⁺ T cells via MHC-presented antigen on the ex vivo infected cells.

In addition, it is contemplated that some of the apheresis product may also be subjected to isolation of NK cells, which may be expanded in vitro to provide additional cytotoxic effector cells. Moreover, and where desired, the isolated NK cells may be further stimulated to differentiate into cytokine enhanced NK cells (CENK) and/or memory-type cytokine enhanced NK cells (m-CENK). These modified NK cells can then be administered to the subject to thereby further augment the immunotherapeutic composition that includes the ex vivo infected apheresis product. Still further, it is contemplated that additional cell-based therapeutics may be administered such as haNK cells or t-haNK cells such as described, for example, in U.S. Pat. No. 11,643,452, and US 2021/0198342.

Moreover, it is contemplated that the apheresis product also comprises erythrocytes and thrombocytes, which may be employed as additional therapeutic agents. As should also be noted, the erythrocytes and/or thrombocytes may be genetically modified to express one or more proteins of interest (e.g., cytokine, checkpoint inhibitor, etc.).

With respect to the administration of the ex vivo infected apheresis composition, it is contemplated that the cells can be transfused to the subject as a stand-alone modality, or in combination with one or more immune stimulating cytokines. Most typically, where the administration also includes transfusion of NK cells (e.g., CENK, m-CENK, CAR-expressing NK cells, etc.), the NK cells will preferably be administered after the ex vivo infected apheresis composition, for example, at least 1 day, or at least three days, or at least seven days, or at least 14 days after transfusion of the ex vivo infected apheresis composition.

Several different treatment options are contemplated using the presently disclosed ex vivo infected apheresis composition. Treatment protocols in addition to the ex vivo infected apheresis composition will include autologous or allogeneic ceNK and/or m-ceNK transplants, autologous or allogeneic blood matched red blood cell transplants, autologous or allogeneic platelet transplants. Moreover, treatment schemes contemplated herein will also include supportive administration of IL-15, IL15: IL15Ra constructs, HCW-9218, or N-803 (subcutaneous or intratumoral) or other cytokines (e.g., NHS-IL-12, see Immunotargets Ther. 2021; 10:155-169), for example, to help proliferate NK cells, T cells, and memory T Cells. Further methods disclosing treatment options can be found in PCT application no.: PCT/US24/26806, which is incorporated by reference herein in its entirety.

In an embodiment, the dendritic cells in the immunotherapeutic composition disclosed herein are transfected with a human adenovirus serotype 5 (hAd5) vector comprising a deletion of the early 1 [E1] and early 2b [E2b] genes, and a nucleic acid encoding at least one immunogenic viral peptide. In this context, it is noted that the term “human adenovirus serotype 5 (hAd5) vector” encompasses a recombinant hAd5 nucleic acid as well as a recombinant hAd5 viral particle containing a recombinant hAd5 nucleic acid.

For example, in some embodiments, the dendritic cells are exposed to the recombinant therapeutic virus (recombinant hAd5 vector encoding at least one immunogenic viral peptide) for a time sufficient for the dendritic cells to take up substantially all of the recombinant therapeutic virus. Among other options, suitable times will be between 60 and 180 minutes, or between 180 and 600 minutes, or between 8 and 12 hours, or between 12-24 hours, and even longer, typically at a MOI of between 1:1 and 100,000 (and even more typically between a MOI of 100 and 50,000. Alternatively, or additionally, residual viruses are removed and reduced in number by replacement of the medium in which the cells are maintained or grown. Thus, it should be appreciated that, upon ex vivo infection, the viral titer in the PBMC supernatant is low to undetectable (e.g., equal or less than 10⁴ viral particles per mL, equal or less than 10³ viral particles per mL, equal or less than 10² viral particles per mL, or even less).

The antigen loaded dendritic cells are exposed to T cells (and especially CD4+ and CD8+ T cells), resulting in a cell population that can be transfused into a subject to elicit an effective immune response against the viral antigen that is encoded by the recombinant adenovirus and expressed in the transfected cells, referred to herein as the virally educated T cells. Alternatively, the ex vivo transfection of the antigen presenting/dendritic cells is performed in the presence of other immune cells such as macrophages, monocytes, T cells, B cells, etc.

In an embodiment, the composition comprises virally educated T cells and N-803. IL-15 may act to facilitate binding between APC-expressed IL15Rα and T cell-expressed IL15Rβγ. N-803 will stimulate expansion of the virally educated T cells.

In an embodiment, the composition is for use in the treatment of infectious disease, particularly long Covid. The Ad5 encoded immunogenic peptide encodes a viral antigen. Identification of an antigenic sequence may derive from sequencing, mRNA expression, and peptide expression analysis.

In a preferred example, fresh or frozen PBMCs obtained from a subject can be treated with GM-CSF and FLT3L for a time sufficient to allow for antigen presenting cells to differentiate (e.g., 8-16 hours, or 12-24 hours, or 18-36 hours, or 24-48 hours), typically also in the presence of Interleukin 4 (IL-4). Subsequently, the so treated cells are then stimulated, optionally in the presence of one or more SARS COV2 peptides (for example N and/or S peptides) or a recombinant virus (e.g., Ad5 virus with recombinant nucleic acid encoding one or more SARS COV2 peptides) or other viral pathogen to so activate the antigen presenting cells. The so differentiated and stimulated cells are then fed, and the T cell sub-population is expanded in the presence of the activated antigen presenting cells using fresh medium containing suitable cytokines (e.g., IL-2, IL-7, IL-15). Most preferably, the so obtained cell population is then once more stimulated (preferably in the presence of anti-CD28, anti-CD3, and/or anti-CD-49d antibodies) with the peptide antigen(s), the inactivated virus, the recombinant virus or other pathogen for at least 6-8 hours. Where desired, the T cells can then be tested for function (e.g., IFN-gamma secretion in a conventional ELISpot assay) prior to transfusion to the subject.

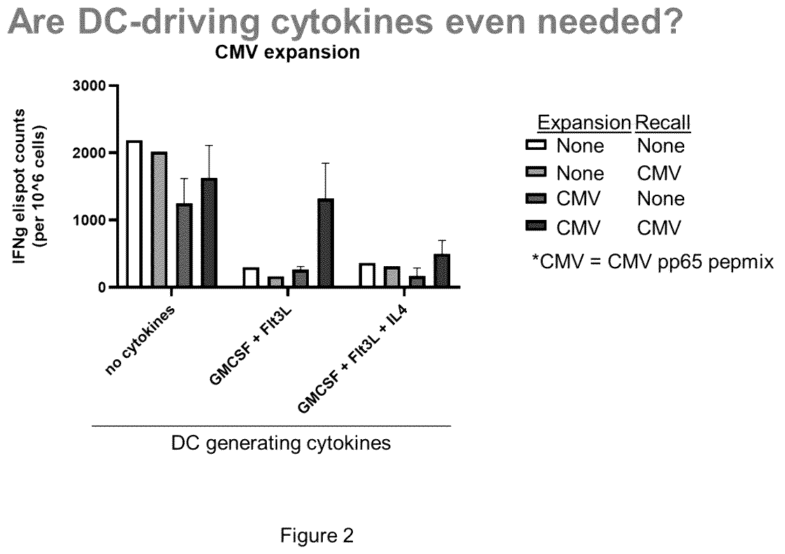

FIG. 2 illustrates that DC-driving cytokines lower overall background and appear to favor specificity. In this example, PBMCs from a CMV pp65 responsive donor are thawed and treated with DC generating cytokine cocktails as listed on Day 0. For Day 1, cells are treated with LPS, R848 (resiquimod; TLR 7/8 agonist) and the CMV pp65 pepmix. At Day 2, T-cell expansion cytokines are added (IL-2, IL-7, IL-15), followed by media exchanges every on days 4 and 7. The cells are harvested on day 9 and tested for reactivity to CMV pepmix using a standard ELISPOT protocol. The data shows that DC-driving cytokines are needed to lower overall background and to favor specificity.

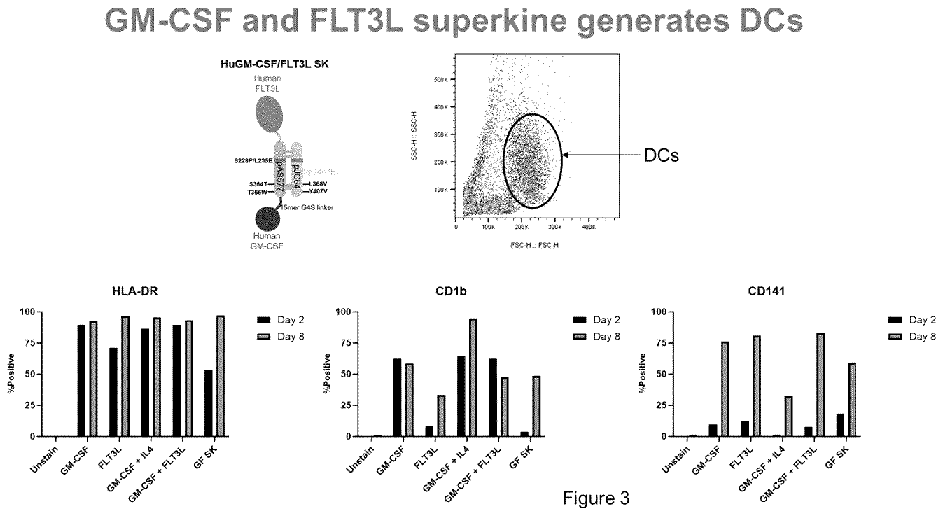

FIG. 3 shows a culture of dendritic cells (DCs) from peripheral blood mononuclear cells (PBMCs) with the superkines GM-CSF and FLT3L. It was found this combination efficiently supported DC expansion and development.

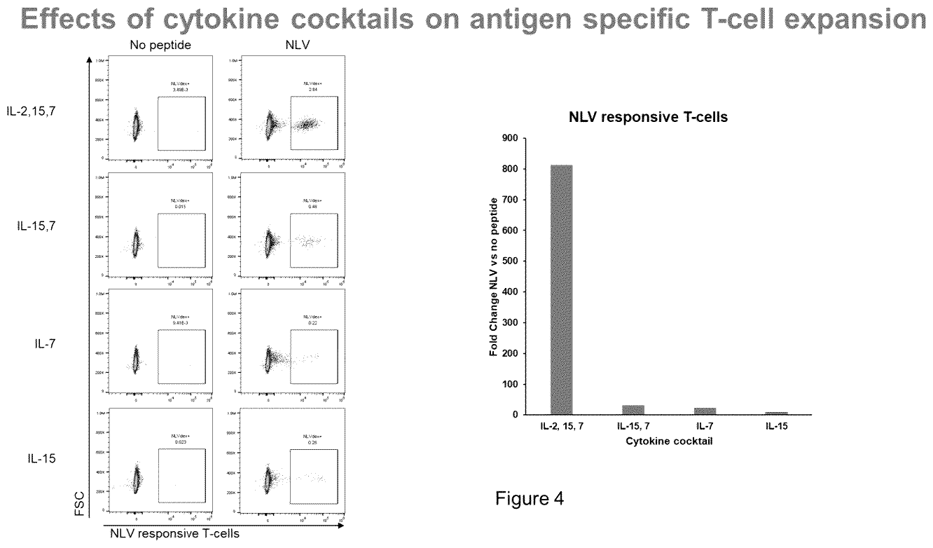

FIG. 4 illustrates the effects of cytokine cocktails on the instant antigen specific T-cell expansion. 5. Cytokines IL-2, IL-7, and IL-15 are known for use in T cell growth, but the optimal cytokine or combination for maximizing this effect remains unclear. The current literature often suggests that IL-2 and IL-15 are equally effective in promoting T cell growth. However, in the presently disclosed composition and method, the inventors observed an unexpected 813-fold increase in NLV-specific T cells when they were expanded with a combination of IL-2, IL-7, and IL-15. Removing IL-2 caused this increase to drop dramatically to only 32-fold, and excluding IL-15 or IL-7 resulted in an even more pronounced decrease.

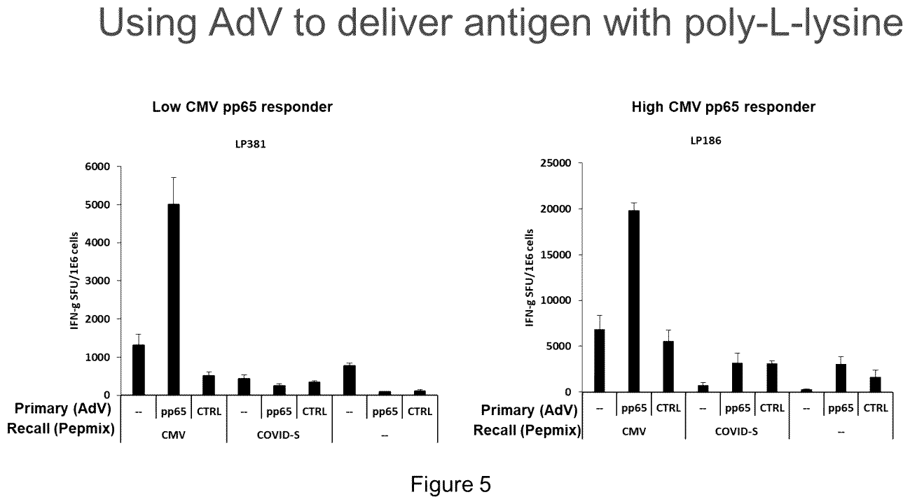

FIG. 5 illustrates the use of an adenovirus to deliver antigen with poly-L-lysine. A low CMV pp65 NLV responder (LP381) and high responder (LP186) were compared, given the disclosed GM-CSF/Flt-3 superkine, AdV expressing pp65 (with poly-L-lysine enhancer), no TLR agonism, IL-2/IL-7/IL-15 T-cell expansion cytokines. The results show that a response to NLV can be effectively and efficiently generated.

In an embodiment, the composition comprises NK cells, wherein the NK cells are purified from the apheresis product. NK cells are screened out by flow sorting or other method. NK cells are identified as CD56+/CD3−. The NK cells are combined ex vivo with an Ad5 vector comprising a nucleic acid encoding at least one Chimeric Antigen Receptor. Optionally, the composition further comprises IL-15, IL-15:IL-15Rα or a derivative thereof, HCW-9218, or N-803. The NK cells may be activated and expanded from PBMC as previously described in U.S. Pat. No. 11,453,862; US 2021/0009954; and/or activated and expanded to produce CIML-NK cells as described in U.S. Pat. No. 20,210,008107; US 2021/0008112; U.S. Pat. No. 20,210,361711; Alternatively, or additionally, NK cells may be differentiated to m-ceNK cells as described in WO/2022/187207. Where the NK cells are transfected with a recombinant nucleic acid to express a CAR, especially contemplated compositions and methods are described in US 2022/0282216. Disclosed compositions are for use in the treatment of an infectious disease. Also disclosed is a method of treatment wherein contemplated compositions are administered IV to a patient in need thereof. The method further comprises subcutaneous administration of IL-15, IL-15:IL-15Rα or a derivative thereof, or N-803 (IL-15 superagonist) to the patient.

In an embodiment, the sorted NK cells are cultured in IL-15 and optionally glucocorticoid, followed by an expansion phase wherein the NK cells are cultured in IL-15/IL-18/IL-12 whereby memory cytokine-enriched natural killer (m-ceNK) cells are induced. See e.g., WO/2022/187207 The m-ceNK cells can be further genetically engineered to express a CAR with specificity for an infectious disease antigen, such as SARS COV2 antigen. The m-ceNK cells can be for autologous administration in combination with the virally educated T cells of the present invention. The m-ceNK cells can be for autologous or allogeneic haploidentical administration, in conjunction with IV administration of the virally educated T cells and N-803 of the present invention.

According to the methods provided herein, the patient is administered an effective amount of one or more of the compositions and agents provided herein for the treatment of long Covid. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 22nd Edition, Gennaro, Editor (2012), and Pickar, Dosage Calculations (1999)).

In a preferred embodiment, the virally educated T cells and/or the ceNK cells described herein is be administered at a dosage of 10⁴ to 10⁹ cells/kg body weight, preferably 10⁵ to 10⁶ cells/kg body weight, including all integer values within those ranges. In preferred embodiments, the dosage of N-803 is about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/kg body weight.

Pharmaceutically acceptable compositions can include a variety of carriers and excipients. A variety of aqueous carriers can be used, e.g., buffered saline and the like. These solutions are sterile and generally free of undesirable matter. Suitable carriers and their formulations are described in Remington: The Science and Practice of Pharmacy, 22nd Edition, Loyd V. Allen et al., editors, Pharmaceutical Press (2012). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject. As used herein, the term pharmaceutically acceptable is used synonymously with physiologically acceptable and pharmacologically acceptable. A pharmaceutical composition will generally comprise agents for buffering and preservation in storage and can include buffers and carriers for appropriate delivery, depending on the route of administration.

The compositions may contain acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of cells in these formulations and/or other agents can vary and will be selected primarily based on fluid volumes, viscosities, body weight and the like in accordance with the particular mode of administration selected and the subject's needs

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A pharmaceutical composition for use in a patient having a viral infection, the composition comprising:
a. IL-15 or an agonist derivative thereof; and
b. Viral-educated T cells comprising T-cells isolated from whole blood of the patient and exposed ex vivo to dendritic cells, wherein the dendritic cells are 1) isolated from whole blood of the patient and 2) transfected with a nucleic acid vector encoding at least one antigenic viral peptide sequence.

2. The composition of claim 1, further comprising memory-like cytokine enhanced natural killer (m-ceNK) cells, wherein the m-ceNK cells are isolated from the whole blood of the patient.

3. The composition of claim 1, wherein the IL-15 agonist derivative is selected from the group consisting of IL-15:IL:15RαSu, IL-15^{N72D}:IL-15RαSu/Fc and N-803.

4. The composition of claim1, wherein the autologous T cells and dendritic cells are derived from an apheresis product.

5. The composition of claim 1, wherein the nucleic acid vector comprises an adenoviral (Ad) vector, and further wherein the Ad vector comprises SARS-COV-2 Spike(S) and nucleocapsid (N) proteins.

6. The composition of claim 5, wherein the Ad vector comprises a first nucleic acid portion and a second nucleic acid portion; wherein the first nucleic acid portion encodes a CoV2 spike (S) protein having the amino acid sequence of SEQ ID NO:3; and wherein the second nucleic acid portion encodes a chimeric protein comprising 1) a CoV2 nucleocapsid (N) protein having the amino acid sequence of SEQ ID NO: 1 and 2) an endosomal targeting sequence (ETSD) having the amino acid sequence of SEQ ID NO: 2.

7. The composition of claim 1, wherein the viral peptide sequence is derived from SARS-COV-2, HPV, or HIV.

8. The composition of claim 1, wherein the T cells are ex vivo exposed to dendritic cells in the presence of IL-15 or an agonist derivative thereof or in the presence of mesenchymal stem cells (MSC).

9. The composition of claim 1 wherein the viral infection comprises SARS-COV-2, or long Covid.

10. A method of treating long Covid in a patient in need thereof, the method comprising:
a. transfecting dendritic cells isolated from whole blood of the patient with a nucleic acid vector encoding at least one antigenic peptide derived from SARS-COV-2;
b. exposing T cells, isolated from the whole blood of the patient, to the dendritic cells, thereby expanding the T cells into viral educated T cells; and
c. treating long Covid by administering IL-15 or an agonist derivative thereof and the viral educated T cells to the patient.

11. The method of claim 10, wherein the nucleic acid vector comprises an adenoviral (Ad) vector.

12. The method of claim 11, wherein the adenoviral (Ad) vector comprises an E1 gene region deletion and an E2b gene region deletion.

13. The method of claim 11, wherein the adenoviral (Ad) vector comprises a nucleic acid portion that encodes a SARS-COV-2 S protein, and a nucleic acid portion that encodes a chimeric protein comprising a SARS-COV-2 N protein and an endosomal targeting sequence.

14. The method of claim 10, wherein the T cells are exposed to the dendritic cells in the presence of IL-15 or an agonist derivative thereof.

15. The method of claim 10, wherein the IL-15 agonist derivative is selected from the group consisting of IL-15:IL:15RαSu, IL-15^{N72D}:IL-15RαSu/Fc and N-803.

16. The method of claim 10, wherein the T-cells are administered intravenously.

17. The method of claim 10, wherein the T-cells and the IL-15 or agonist derivative thereof are administered simultaneously.

18. The method of claim 10, further comprising administering Natural Killer (NK) cells to the patient.

19. The method of claim 18, wherein the NK cells are selected from the group consisting of comprise m-ceNK cells isolated from the whole blood of the patient, NK-92 cells, aNK cells, haNK cells, and t-haNK cells.

20. The method of claim 10, wherein the SARS-COV-2 antigenic peptide comprises a CoV2 spike(S) protein having the amino acid sequence of SEQ ID NO:3, or a CoV2 nucleocapsid (N) protein having the amino acid sequence of SEQ ID NO: 1, or fragments thereof.


SEQUENCE LISTING (3 sequences)

SEQ ID NO: 1 - protein, 473 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGPGPGNLVPMVATVGPGPGMLIPIAVGGALAGLVLIVLIAYLIGKKHCSYQDIL

SEQ ID NO: 2 - protein, 27 aa, synthetic construct
MLLLPFQLLAVLFPGGNSEDYKDDDDK

SEQ ID NO: 3 - protein, 1282 aa, synthetic construct
MFVFLVLLPLVSSYPYDVPDYAQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT